(19) United States
(12) Patent Application Publication
(10) Pub. No.: US 2022/0142503 A1
(43) Pub. Date: May 12, 2022

(54) Gastrointestinal capsule and method

(71) Applicant: Xiaoning Huai, Sunnyvale, CA (US)

(72) Inventor: Xiaoning Huai, Sunnyvale, CA (US)

(73) Assignee: Real Image Technology Co., Ltd, Shenzhen (CN)

(21) Appl. No.: 17/520,669

(22) Filed: Nov. 7, 2021

Related U.S. Application Data
(63) Continuation-in-part of application No. 17/372,518, filed on Jul. 12, 2021.

(30) Foreign Application Priority Data
Aug. 11, 2021 (CN) 2021109174009
Nov. 3, 2021 (CN) 2021112912234
Nov. 3, 2021 (CN) 2021112953022
Nov. 3, 2021 (CN) 2021226702773
Nov. 3, 2021 (CN) 2021226741119

(51) Int. Cl. A61B 5/07; A61B 5/00

(52) U.S. Cl. CPC A61B 5/073; A61B 5/4255

(57) ABSTRACT

The invention discloses a bidirectional ultrasonic capsule for gastrointestinal measurement. The capsule comprises pairs of ultrasonic ranging probes to obtain correlated depth or morphology data of two sides of an inner wall of a digestive tract, wherein Probe 1 is configured to range a first distance to a first side of the inner wall of the digestive tract along a first direction and Probe 2 is configured to range a second distance to a second side of the inner wall of the digestive tract along an opposite direction to the first direction, and data of the first and second distances are correlated and a sum of the first distance, the second distance and a third distance between Probe 1 and Probe 2 is obtained as a directional cavity diameter of the digestive tract, which eliminates errors caused by motion of the capsule in the digestive tract in the unidirectional measurement of the prior art.

### TECHNICAL FIELD

The invention relates to the technical field of medical devices, in particular to gastrointestinal motility and capsule.

### BACKGROUND

Gastrointestinal motility is important to human physiology and pathology. The measurement of gastrointestinal motility in the prior art is mainly based on the tracking of radioactive markers, as disclosed in U.S. patent application Ser. No. 15/881,671. Because radiation examination is harmful to organisms, the basic research and clinical application of gastrointestinal motility need a non-invasive testing scheme in vivo. Light, sound and magnetism are commonly used noninvasive testing vehicles. The 3D camera and gastrointestinal capsule robot with magnetic positioning such as EndoCapsule 10 system of Olympus have been commercially available, which provides a good technical feasibility for the scheme of the invention. The capsule robot can include sensors, controllers and intelligent processors. The sensor and at least part of the controller are usually located in the capsule, the intelligent processor is usually located in an external control terminal, and the sensor, the controller and the intelligent processor are usually connected by wired or wireless communication links. Due to the extensive commercial application of the capsule robot, the implementation of the capsule robot well known to ordinary skill in the art will not be elaborated in the following description of the invention.

### SUMMARY OF THE INVENTION

The invention provides a 3D ultrasonic capsule for measurement of morphologies of the digestive tract, and the capsule is configured to obtain the directional cavity diameter of the inner wall of the digestive tract using a plurality of pairs of reversely positioned ultrasonic ranging probes which form a spherical array of ultrasonic ranging probes. The endpoints of each directional cavity diameter are located on the opposite inner walls of the digestive tract and contains a pair of data of the depth map. The capsule contains a magnet for positioning the pose and position of the capsule, and the depth data of the inner wall of the digestive tract are collected in a target area in the digestive tract.

The invention provides a first method for measurement of gastrointestinal motility, comprising the following steps:

obtaining one or more of depth, morphology and image data of the inner wall of digestive tract; obtaining data of the surface of the inner wall of digestive tract;

extracting the morphological features of the surface data, wherein the morphological features include one or more of the anatomical parts of the inner wall of the digestive tract, curvature, inner diameter and volume.

The invention also provides a second method for measurement of gastrointestinal motility, comprising driving a capsule to a target area of digestive tract and applying an intervene magnetic force on the capsule by a magnetron;

obtaining data of a first transit time of the capsule when no magnetic force is applied on the capsule;

obtaining data of a second transit time of the capsule when a first magnitude of a magnetic force is applied on the capsule and the difference between the first transit time and the second transit time is bigger than a threshold;

obtaining data of a second set of transit time of the capsule when the magnetic force is increased from the first magnitude to a second magnitude wherein transit of the capsule is blocked;

conducting an evaluation of gastrointestinal force based on the data of the first and second magnitude of the magnetic force, the first transit time and the second set of transit time, physical characteristics of the capsule and physical characteristics of gastric contents.

The invention provides a gastrointestinal motility measurement system based on a gastrointestinal capsule, which comprises a data acquisition module, a data processing module and a capsule. The data acquisition module and the data processing module are connected by a wired or wireless communication link. The data acquisition module is configured in the capsule, and comprises an ultrasonic distance measuring device or a camera for acquiring one or more of depth, morphology and image data of the inner wall of the digestive tract. The data processing module is preferably set in a control terminal outside the body, or in a distributed manner, wherein part of the functions is completed in the control terminal and part of the functions are completed in the capsule. The data processing module has at least one processor and at least one non-volatile storage medium, wherein the non-volatile storage medium contains instructions and parameters that can be read by the at least one processor, causing the at least one processor to run a digestive tract motility measurement program which coordinates the different modules. The data processing module is used to process the one or more of depth, morphology and image data to extract morphological features, including position, curvature, inner diameter and volume which are used as references for evaluation of gastrointestinal motility.

The invention provides another gastrointestinal motility measurement system, which comprises a control module, a magnetic driving module, a magnetic positioning module and a capsule. The control module, the magnetic driving module and the magnetic positioning module are connected by a communication link. The capsule is provided with a positioning magnet and a driving magnet, which could be a single magnet or two separate magnets. The positioning magnet generates a magnetic field signal, which is detected by the magnetic positioning module obtaining the position and motion data of the capsule in the digestive tract relative to an external coordinate system. The magnetic driving module generates a driving magnetic field, and the driving magnetic field acts on the driving magnet of the capsule to generate a driving magnetic force to drive the capsule to move in the digestive tract. The control module obtains a first position and motion data of the capsule under the action of gastrointestinal motility through a magnetic positioning module; obtains the second position and motion data of the capsule under the joint action of the gastrointestinal motility and the driving magnetic force. The gastrointestinal motility is estimated according to the first and second position and motion data and the driving magnetic force.

### BRIEF DESCRIPTION OF THE FIGURES

FIG. 1 is a schematic diagram of gastric peristalsis. It shows the morphological changes of gastric wall during peristalsis in the order from 1 to 4, wherein the inner diameter and curvature of the digestive tract flips between convex and concave during the gastrointestinal peristalsis.

FIG. 2 is an illustration of an ultrasonic capsule operation.

FIG. 3 is an illustration of the System Diagram.

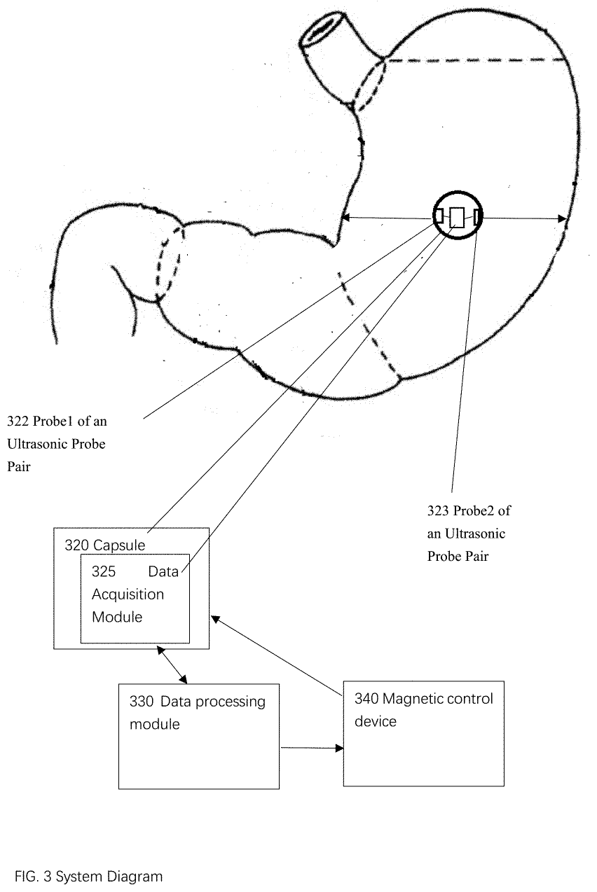

FIG. 4 is an illustration of the components in a spherical capsule.

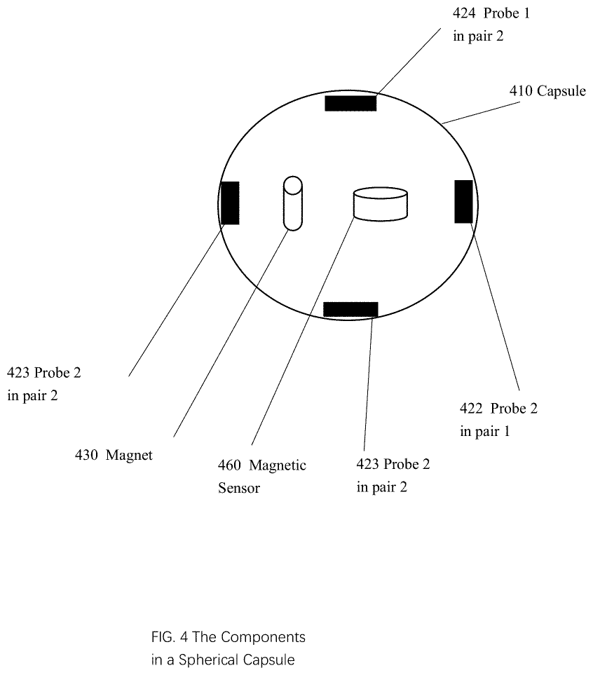

FIG. 5 is an illustration of spherical Probe Array in an oval capsule.

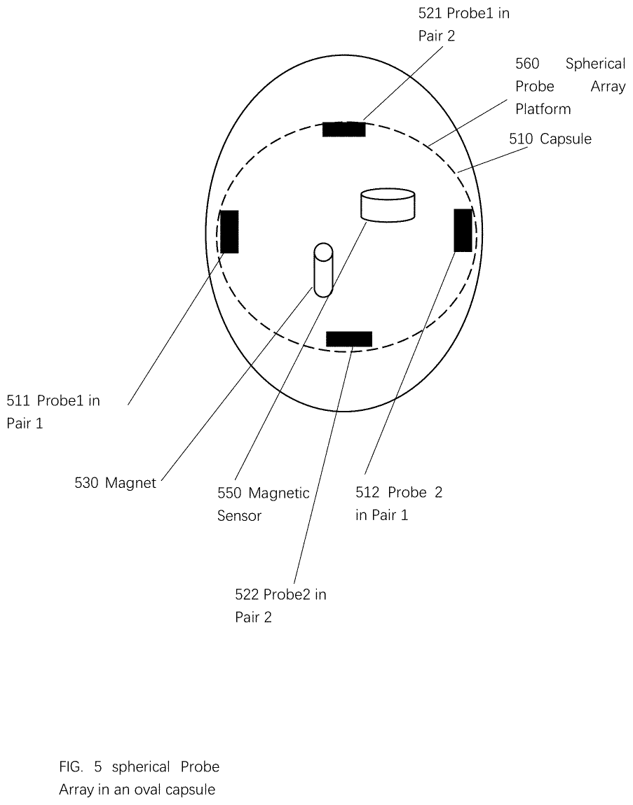

### DETAILED DESCRIPTION

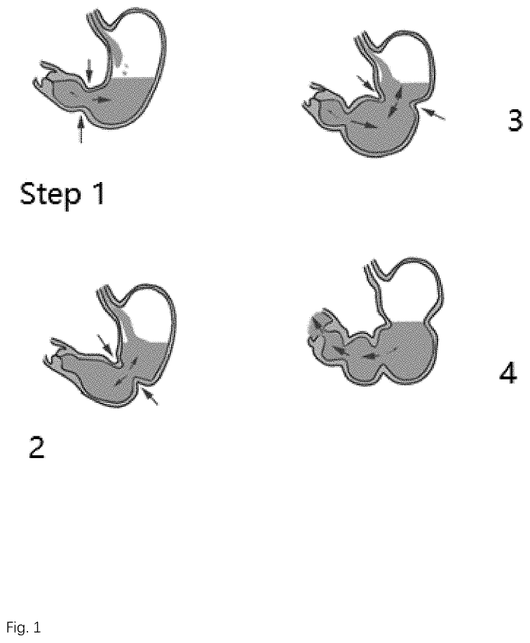

Gastrointestinal motility generally refers to the force and frequency of gastrointestinal contraction, relaxation and peristalsis under the action of gastrointestinal muscles. Its function is to make food move and be transmitted, so as to be digested, absorbed and emptied. An intuitive view of the relationship between the morphological characteristics of the digestive tract and the gastrointestinal motility comprises that under the action of the digestive tract muscles, the gastrointestinal peristalsis first produces deformation, including the change of the curvature of the digestive tract and the change of the inner diameter of the digestive tract. The deformation then transfers the force of the digestive tract muscle to the contents of the digestive tract, such as chyme, so as to make the contents of the digestive tract. Second, the digestive tract, like most other tissues in the human body, can be elastic. It is well known that the force on an elastic body is proportional to the deformation of the body under the force. Therefore, there is a close correlation between the morphological changes of the digestive tract and the gastrointestinal motility. As shown in FIG. 1, the changes in the inner diameter and curvature of the digestive tract include the frequency of the convex and concave flips of the surface of the inner walls of the digestive tract are directly correlated with the frequency and intensity of gastrointestinal peristalsis, which can be based on to determine the frequency and intensity of gastrointestinal motility. On the other hand, there are significant morphological differences in physiology and pathology of gastrointestinal peristalsis. For example, when stenosis, dilation or obstruction occurs, the normal rhythm of contraction and relaxation will change. Through statistical analysis of the data of the morphological characteristics and the changes of the morphological characteristics and the frequencies of the changes of concerned areas of the digestive tract, a model of the morphological and dynamic characteristics of the digestive tract can be obtained, which can be used as a reference for evaluating the gastrointestinal motility. Like curvature and inner diameter, the morphological characteristics of digestive tract also include the change of volume of target areas of gastrointestinal lumen during peristalsis. The change of volume reflects the emptying amount of gastrointestinal peristalsis, which is related to the work done by gastrointestinal muscles and the energy produced.

The characteristic parameters of the digestive tract proposed above by the invention can preferably be acquired by first obtaining the depth map or point cloud of the inner wall of the digestive tract. Then the morphological features are extracted. Specifically, an ultrasonic distance measuring device can be preferably set in the capsule. After the capsule enters the body, the ultrasonic distance measuring device is started to obtain the distance from the capsule to the inner wall surface of the digestive tract. The ultrasonic measurement device can also collect the distance from the capsule to the multi-layer tissue structure of the inner wall of the digestive tract. Ultrasonic ranging mainly uses time difference ranging method. An ultrasonic probe emits directional ultrasonic wave and starts a timer at the same time of transmitting. The timer is stopped when the probe receives the reflected wave. Let V be the propagation velocity of the ultrasonic wave in the medium, T be the time difference between the transmitted wave and the returned wave recorded by the timer, and S be the distance from the transmitting point to the reflecting point

S=V×T/2

Let the capsule be of a sphere shape, the center of which is located at a point in the digestive tract lumen. The sum of the distance from the point to a point on the inner wall of the digestive tract in an arbitrary direction and the distance from the point to a point on the inner wall of the digestive tract in the opposite direction is defined as the directional cavity diameter of the digestive tract in the present invention. The directional cavity diameter is a measurement of the geometric size of the inner wall of the digestive tract by the ultrasonic ranging device, and also includes a pair of sampling points of the depth map of the inner wall of the digestive tract. There are multiple directional cavity diameters passing through any point. The spatial resolution of the depth map or point cloud and the final surface of the inner wall of the digestive tract is determined by the sampling interval, which conforms to the Nyquist law. A plurality of ultrasonic ranging probes can be preferably set in the capsule to form a spherical distribution ultrasonic ranging probe array platform including mechanism, circuit and control software, which is used to obtain multi-directional or panoramic depth map or point cloud data. Obviously, the denser the probe array, the more sampling points, and the higher the corresponding cost and circuit power consumption. Or a mechanical rotation device can be set on the platform of a sparse probe array, and it may rotate an angle after one sampling, and then conduct the next sampling. The platform comprises the following characteristics when conducting one measurement: First, all probes are located on a spherical surface; and second, the ranging directions of the two probes of any pair of probes are opposite yet correlated, and the connecting lines of the ranging directions of the two probes preferably pass through the ball center; and thirdly, the measurements by two probes of a pair are synchronized.

As the capsule is in a transit under the gastrointestinal peristalsis, the depth map or point cloud data from multiple sampling may preferably be matched, registered and fused. In addition to ultrasonic ranging device, 3D camera based on infrared or visible light sensor can also be used to obtain panoramic depth map or point cloud.

With the peristalsis of the alimentary tract, the capsule moves passively and randomly in the alimentary tract, and is finally discharged from the body. A preferred implementation of the invention can use the magnetic field generated by the magnetic control device to drive the capsule with a magnet in it to move in the digestive tract, or hold the capsule to stay in a target area for a measurement in-situ. Another preferred implementation of the invention is for the capsule to work intermittently, which is used to reduce the power consumption of the capsule battery.

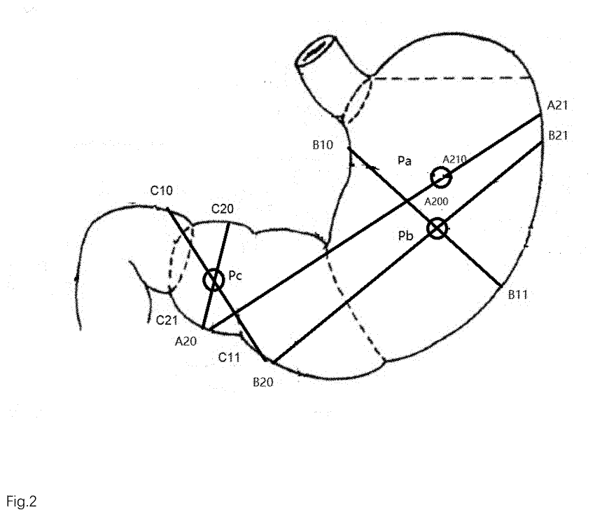

FIG. 2 is an example of an ultrasonic capsule operation. After the capsule enters a subject's body, it can get to a point Pa first. Probe 1 of a probe pair located at a point on an exterior wall of the capsule A210 takes a measurement of the distance to a point A21 on the gastric wall along an arbitrary direction of (θ, φ) in a spherical coordinate system with its coordinate origin set at Pa, wherein the distance is expressed by |a210, A21|. At the same time or in a synchronized manner, Probe 2 located at A200 on the opposite side of the capsule takes a measure of the distance between A200 to a point A20 on the gastric wall along the opposite direction (−θ, −φ), wherein the distance is expressed by |A200, A20|. The distance of |A210, A21|+|A200, A20|+|A200, A210| is a directional cavity diameter D passing through point Pa. Coordinates (θ, φ, |A210, A21|+1/2*|A200, A210|) and (−θ, −φ, |A200, A20|+1/2*|A200, A210|) are a pair of depth data of a depth map obtained by the capsule at point Pa. The collection of the depth data of all points of gastric wall acquired by the capsule at point Pa is the depth map at point Pa. The depth map obtained from different points, such as Pb, Pc, can be matched and fused into a depth map, and then the depth map can be transformed into a point cloud, or each depth map can be transformed into a point cloud, and then the point cloud can be matched and fused. Magnetic positioning may preferably be used to track and mark the pose and position of the capsule as a parameter for depth map or point cloud fusion. The point cloud can be regarded as a sample of the inner surface of digestive tract. Sparse point clouds can be smoothed and denoised by surface fitting algorithms to obtain surface data. With the peristalsis of the alimentary canal, the surface data of the inner wall of the whole alimentary canal can be accumulated. Because different parts of the human digestive tract have unique local morphological characteristics and corresponding relationship, the data processing module can recognize local morphological characteristics of the digestive tract through machine learning or other techniques. In an example to take a measure of an area of interest, such as a point Pc in FIG. 2, assuming the current position of the capsule being at a point Pa, the magnetic control device can be started to drive the capsule from point Pa to point Pc. When the magnetic positioning device confirms that the capsule has reached point Pc, the system control software of the data processing module starts the ultrasonic ranging device of the capsule to collect data. Furthermore, the data processing module will match the current pose and position data of the capsule collected in real time by magnetic positioning with the pose and position data obtained from analysis of the data of the inner wall of the digestive tract collected by the capsule to ensure the accuracy of the positioning. During a motility test, it may be optimized to minimize the perturbation of the test on the surrounding physiological environment, such as the design of the capsule of a small volume and with a round shape, a sleek shell of the capsule body, and a close density to that of chyme. In a test without intervention, the driving force of the magnetic control equipment can usually be in the zero state. In an intervention test, intervention force can be applied to maintain the capsule in an area of concern, or the capsule motion can be obstructed so he gastrointestinal force in the balance can be measured. As an embodiment, the capsule is observed at point Pc, near the pylorus. When the magnetic force reaches a first threshold, the transit time of the capsule increases. When the magnetic force reaches a second threshold, the capsule can not be emptied. The peristaltic force of the capsule can then be estimated according to the transit time, the magnitude and direction of the magnetic force, the physical characteristics of the capsule and the physical characteristics of the gastric contents. After obtaining the depth map of the inner wall of digestive tract from the time series collected by the capsule, the data processing module can first convert the depth map into point cloud, and then perform surface fitting. Since the main function of the digestive tract is to move around the food, the direction of food motion can be regarded as the principal axis direction or the principal transit direction of the digestive tract. A statistical average value of a plurality of directional cavity diameters perpendicular to the principal axis at a point of concern in the digestive tract can be set as an inner diameter of the digestive tract at that point. According to the surface data and the anatomic characteristics of digestive tract, the path of the principal transit connecting the points in the digestive tract can be estimated. The calculation of curvature of a surface is a classic subject of differential geometry, and there are a large number of algorithms to choose from. For volume calculation, a length-adjustable line segment (L1, L2) can be selected along the direction of the principal transit as a height, where L1 and L2 are the coordinates of the end points. Through L1 and L2, the vertical plane S1 and S2 in the direction of principal transit are made respectively. A closed body surrounded by surface data of plane S1, S2 and the surface of inner wall of digestive tract can be regarded as a volume at point Pc, which can be calculated by integral numerical method. The motion data of the capsule, including displacement, velocity and frequency, can be obtained by magnetic positioning device. The change rate and range of the above gastrointestinal morphological features can be extracted from the time series data, and the frequency characteristics can be correlated with the frequency characteristics of the capsule motion. Different foods or drugs can affect gastrointestinal motility. The above tests can be carried out in food environment such as water, starch and wine.

What is claimed is:

1. A capsule, comprising one or more pairs of ultrasonic probes configured to obtain correlated data of two sides of a wall of a region of a digestive tract.

2. The capsule of claim 1, wherein each of the pairs comprises a first probe and a second probe, the first probe configured to range a first distance to a first side of the wall of the region of the digestive tract along a first direction and the second probe configured to range a second distance to a second side of the wall along an opposite direction to the first direction; an angle between the first and second directions is smaller than a threshold.

3. The capsule of claim 1, wherein the capsule or a first apparatus linked to the capsule through communication is configured to obtain morphological features from the data.

4. The capsule of claim 3, the morphological features comprising one or more of location, volume, curvature and diameter of the region.

5. The capsule of claim 3, wherein the capsule or the first apparatus or a second apparatus is configured to obtain parameters of motility of the digestive tract referencing the morphological features.

6. The capsule of claim 5, the parameters comprising frequency and strength of peristalsis of the digestive tract.

7. The capsule of claim 4, wherein the capsule or the apparatus is configured to obtain the diameter by adding the first distance, the second distance and a distance between the first and second probes.

8. A capsule, comprising one or more pairs of ultrasonic probes, each of the pairs consisting of a first probe and a second probe; the first probe is configured to range a first distance to a first side of a wall of a region of a digestive tract along a first direction; the second probe is configured to range a second distance to a second side of the wall along an opposite direction to the first direction; an angle between the first and second directions is smaller than a threshold; the capsule is configured to obtain data of the first and second distances.

9. The capsule of claim 8, wherein the capsule or a first apparatus linked to the capsule through communication is configured to obtain morphological features from the data.

10. The capsule of claim 9, the morphological features comprising one or more of location, volume, curvature and diameter of the region.

11. The capsule of claim 9, wherein the capsule or the first apparatus or a second apparatus is configured to obtain parameters of motility of the digestive tract referencing the morphological features.

12. The capsule of claim 11, the parameters comprising frequency and strength of peristalsis of the digestive tract.

13. The capsule of claim 8, comprising an array of ultrasonic probes distributed on a spherical surface platform.

14. The capsule of claim 8, wherein a magnetic control device is configured to drive the capsule to the region.

15. A method of gastrointestinal motility measurement, comprising the steps of:
obtaining morphological features of a wall of a region of a digestive tract by a capsule;
obtaining parameters of motility of the digestive tract referencing the morphological features.

16. The method of claim 15, the morphological features comprising one or more of curvature, diameter, volume and location of the region.

17. The method of claim 15, the parameters comprising frequency and strength of peristalsis of the digestive tract.

18. The method of claim 16, the obtaining the diameter comprising the steps of:
driving the capsule to the region;
obtaining a principle axis of the region;
obtaining a plurality of directional cavity diameters perpendicular to the principle axis;
and obtaining the diameter of the region by averaging values of the plurality of the directional cavity diameters.

19. The method of claim 17, the obtaining one of the plurality of the directional cavity diameters comprising the steps of:
obtaining a first distance from a first ultrasonic probe of the capsule to a first side of the wall along a first direction;
obtaining a second distance from a second ultrasonic probe of the capsule to a second side of the wall along an opposite direction to the first direction;
obtaining a sum of the first distance, the second distance and a distance between the first and second probes, wherein an angle between the first direction and the second direction is smaller than a threshold.

20. The method of claim 16, wherein the obtaining the volume comprising the steps of:
driving the capsule to the region;
acquiring a surface of the wall of the region;
acquiring a principal axis of the region;
acquiring a segment along the principal axis,
obtaining two planes vertical to the principal axis, each comprising coordinates of an endpoint of the segment;
obtaining the volume of the region enclosed by the two planes and the surface.
